(19) United States
(12) Patent Application Publication
(10) Pub. No.: US 2009/0069223 A1
(43) Pub. Date: Mar. 12, 2009

(54) Method for treatment of vascular hyperpermeability

(76) Inventors: Ed. W. Childs, Temple, TX (US); W. Roy Smythe, Belton, TX (US)

Correspondence Address: STRASBURGER & PRICE, LLP, 901 MAIN STREET, SUITE 4400, DALLAS, TX, 75202, US

(21) Appl. No.: 12/229,127

(22) Filed: Aug. 20, 2008

Related U.S. Application Data
(60) Provisional application No. 60/965,586, filed on Aug. 21, 2007.

(51) Int. Cl. A61K 38/18; A61K 31/381; A61K 38/13; A61P 7/00

(52) U.S. Cl. 514/11; 514/12; 514/440

(57) ABSTRACT

A method for the treatment of apoptotic-induced vascular hyperpermeability is disclosed herein. Administration of various compounds including intrinsic mitochondrial regulatory proteins, certain pharmaceuticals, antioxidants and endothelial growth factors alone or in combination results in elevating the threshold for apoptosis in patients with hemorrhagic shock. Elevating the threshold for apoptosis in patients with hemorrhagic shock decreases the amount of vascular hyperpermeability exhibited by the injured patient. Decreasing the amount of vascular hyperpermeability in a traumatized patient facilitates resuscitation and recovery from trauma and decreases the mortality and morbidity rate in injured patients.

### CROSS-REFERENCE TO RELATED APPLICATIONS

This utility patent application claims priority to and benefit of provisional application No. 60/965,586 filed on Aug. 21, 2007.

### STATEMENT REGARDING FEDERALLY SPONSORED RESEARCH OR DEVELOPMENT

The invention disclosed herein was supported in part by Grant No. 5K01HL76815-3 from the National Institutes of Health and Grant No. HL-03-011 from the National Institutes of Health.

### REFERENCE TO A SEQUENCE LISTING

N/A

### BACKGROUND OF THE INVENTION

1. Field

The invention disclosed herein is a method for treatment of vascular hyperpermeability as a consequence of hemorrhagic shock.

2. Description of the Related Art Including Information Disclosed Under 37 CFR 1.97 and 37 CFR 1.98

Trauma is a leading cause of death for individuals under the age of 44. Individuals who have suffered extensive trauma exhibit hemorrhagic shock which is usually treated with fluids and medicines to maintain blood pressure. Despite the best efforts, patients die because of the inability to maintain sufficient blood pressure to properly perfuse the major organs of the body. One of the causes of death is vascular hyperpermeability secondary to hemorrhagic shock. Vascular hyperpermeability is the process by which the fluid portion of blood leaks out of the vascular structures into the tissues of the body. This leakage of fluid causes the tissues to swell or develop edema. Fluid congestion of the tissues and organs may develop which can result in organ failure. Vascular hyperpermeability also causes some of the fluid administered intravenously during resuscitation efforts to leak out of the vascular system into the surrounding tissues. This leakage of the intravenous fluids from the vascular system contributes to the edema and organ failure. Leakage of intravenous fluids also makes it difficult to maintain an effective blood pressure and perfusion of the organs with oxygenated blood.

Apoptosis or programmed cell death is a normal process in which old cells die and are replaced with new cells. Apoptosis is an orderly process of cell death as distinguished from necrosis which is the result of acute cellular injury. In the body, cells are constantly dying and being replaced. Cells die when they are damaged beyond repair, infected with a virus or undergoing stress such as starvation. These cells are removed and replaced with new cells. In some circumstances, the balance between old cell death and new cell division is out of balance. When cell division is faster than cell death, tumors develop. When cell division is slower than cell death a disorder in the structure and function of the affected tissue occurs.

The process of apoptosis is managed by extracellular signals and intracellular signals. The extracellular signals may include hormones, growth factors or cytokines which must cross the cell membrane in order to effect a response. The intracellular signal may be initiated by a cell under stress and result in cell death. Before cell death can occur the signals mentioned above must be connected to the actual death pathway by way of regulatory proteins. There are several proteins involved in regulation of apoptosis. One set of proteins target the mitochondria. It is these proteins which will be discussed below. That portion of the apoptotic process which targets the mitochondrion is frequently referred to as “intrinsic apoptosis”.

The mitochondrion is a cell organelle which is essential to the life of the cell. The main function of mitochondria is to enable aerobic respiration or energy production by the cell. Disruption of the mitochondrion quickly results in cell death. The apoptotic regulatory proteins affect the permeability of the mitochondrion to cause swelling of the mitochondrion through the development of pores in the membrane of the mitochondria. Cytochrome c is released from the mitochondria due to the increased permeability of the outer mitochondrial membrane and serves a regulatory function as it precedes morphological change in the cell associated with apoptosis. Once cytochrome c is released, it binds with another regulatory protein and ATP, which then binds to pro-caspase-9 to create an apoptosome. The apoptosome cleaves the pro-caspase to its active form of caspase-9, which in turn activates the effector, caspase-3. Caspase-3 is an enzyme which cleaves other proteins to actually start the process of intrinsic apoptosis.

Mitochondrial permeability is subject to regulation by various proteins of the Bcl-2 family of proteins. The Bcl-2 proteins are able to promote or inhibit apoptosis by either direct action on mitochondrial permeability or indirectly through other proteins. Some of the Bcl-2 proteins can stop apoptosis even if cytochrome c has been released by the mitochondrion. The Bcl-2 proteins are frequently referred to as intrinsic mitochondrial regulatory proteins.

Hemorrhagic shock and resuscitation activates a cascade of inflammatory mediators, resulting in tissue damage, multiple organ dysfunction, and if unabated, death. Ischemia associated with shock and the resulting oxidative stress during resuscitation contribute to the development of this systemic inflammatory response. The oxidative stress caused by ischemia/reperfusion results in an increase in reactive oxygen species (ROS) generation which activates leukocytes and damages endothelial cells. Activation of ROS that subsequently damages the endothelium has been shown to increase microvascular permeability. It has been demonstrated that ROS are generated following hemorrhagic shock. Tharakan et. al. Shock, in press. In addition, it has been shown that the endothelium is an important source of ROS generation. Since ROS are by-products of oxidative phosphorylation, most intracellular ROS are produced by the mitochondria. ROS produced at sites other than mitochondria have been reported to be involved in some apoptotic systems, but it is widely accepted that the mitochondrion are the predominant source of ROS produced in the “intrinsic” mitochondrial apoptotic cascade.

Apoptosis can also be regulated by certain cell-specific growth factors. For example, the endothelial cell growth factor, angiopoietin-1, has been observed to stop apoptosis and prevent vascular hyperpermeability and edema following hemorrhagic shock. The angiopoietin-1 prevents apoptosis of endothelial cells by regulating the apoptotic signaling pathway leading to endothelial cell death and vascular hyperpermeability. Childs et. al. Am J. Physiol Heart Circ Physiol 294:H2285-2295. 2008. Treatment of traumatized animals with angiopoietin-1 shows that this compound is a potent inhibitor of vascular hyperpermeability and apoptosis.

If apoptosis continues to cell death, several morphological features are evident:

1. Cell shrinkage and rounding due to the breakdown of the proteinaceous cytoskeleton by enzymes.
2. The cytoplasm of the cell appears dense, and the organelles appear tightly packed.
3. Chromatin undergoes condensation into compact patches against the nuclear envelope.
4. The nuclear envelope becomes discontinuous and the DNA inside is fragmented.
5. The cell membrane shows irregular buds or blebs.
6. The cell breaks apart into several apoptotic bodies which are removed by phagocytosis.

By this process the dead and dying cells and their contents are removed in an orderly fashion and replaced with new, viable cells.

There are currently no available methods or treatments to inhibit apoptosis of endothelial cells following trauma and shock. The ability to inhibit apoptosis of endothelial cells following shock would diminish conditions such as edema caused by vascular hyperpermeability resulting from the death of the endothelial cells. What is needed in the art is a method to protect the endothelial cells from apoptotic death and prevent edema from developing after the patient has suffered trauma sufficient to induce hemorrhagic shock and vascular hyperpermeability.

### BRIEF SUMMARY OF THE INVENTION

The invention disclosed herein is a method to protect the endothelial cells from apoptotic death and prevent edema from developing after the patient has suffered trauma sufficient to induce hemorrhagic shock and vascular hyperpermeability. The method for treatment of vascular hyperpermeability includes administration of a variety of compounds, either alone or in combination, which increases the threshold for intrinsic apoptosis in the endothelial cells of the body. By increasing the threshold for intrinsic apoptosis in the endothelial cells of the body, cell death resulting in vascular hyperpermeability is avoided or minimized. Some of the compounds which modulate apoptosis, either alone or in combination, are shown in the following table:

| Intrinsic Regulatory | | | |
|---|---|---|---|
| Proteins | Pharmaceuticals | Antioxidants | Other |
| Bcl-2 Proteins | Cyclosporine A | a-lipoic acid | Angiopoietin - 1 |
| Bcl-2 | FK 506 | Deprenyl | Estradiol |
| Bcl-xl | | | |
| MCI-1 | | | |
| A1 | | | |
| Bcl-w | | | |
| Non Bcl-2 Proteins | | | |

Some of the diseases or conditions which are associated with vascular hyperpermeability associated with apoptosis are shown in the following table:

| | | | Degenerative | | |
|---|---|---|---|---|---|
| Trauma | Infection | Inflammation | Disease | Edema | Other |
| Trauma | Meningitis | Pneumonitis | Dementia | Brain | ARDS |
| to any | Hepatitis | Non- | Alzheimer's | Heart | Post |
| organ | Pneumonia | infectious | Parkinson's | Secondary to | pneumonectomy |
| | Anthrax | Hepatitis | | Chemotherapy | Syndrome |
| | Myocarditis | | | Liver | Acute Radiation |
| | | | | | Poisoning |
| | | | | | Abdominal |
| | | | | | Compartment |
| | | | | | Syndrome |
| | | | | | Ischemia |
| | | | | | Reperfusion |
| | | | | | Injury |
| | | | | | Anasarca |

### BRIEF DESCRIPTION OF THE SEVERAL VIEWS OF THE DRAWINGS

A better understanding of the invention disclosed herein may be had by review of the following drawings wherein:

FIG. 1 is a bar graph showing the attenuation of hemorrhagic shock-induced vascular hyperpermeability by Bcl-xl administered before, during and after the onset of shock;

FIG. 2 is a graph showing the attenuation of hyperpermeability induced by Bcl-xl given during resuscitation following 60 minutes of shock;

FIG. 3 is a graph showing the attenuation of hyperpermeability induced by Bcl-xl given during the shock period;

FIG. 4 is a graph showing the attenuation of hyperpermeability induced by Bcl-xl when given prior to the induction of shock;

FIG. 5 is a bar graph showing the diminution in release of cytochrome c following administration of Bcl-xl;

FIG. 6 is a bar graph showing the diminution in hemorrhagic shock-induced caspase-3 activity by Bcl-xl administration;

FIG. 7 is a graph showing the elimination of vascular permeability by the administration of cyclosporin A prior to the onset of hemorrhagic shock;

FIG. 8 is a bar graph showing the diminution in cytochrome c release following the onset of hemorrhagic shock by administration of cyclosporin A;

FIG. 9 is a bar graph showing the diminution in hemorrhagic shock-induced caspase-3 activity by administration of cyclosporin A.

### SUMMARY OF THE PRESENT INVENTION

The invention disclosed herein describes a method for treatment of vascular hyperpermeability and prevention of edema in trauma patients by attenuation of the process of apoptosis of endothelial cells lining the structures of the vascular system. Attenuation of apoptosis disclosed in the present invention would have the effect of decreasing vascular hyperpermeability by protecting the endothelial cell barrier from leakage of fluid into the interstitial space caused by apoptosis. It is the intrinsic mode of apoptosis governed by the mitochondria which is being manipulated to reduce vascular permeability and edema associated with hemorrhagic shock.

As described above, the Bcl-2 family of proteins are important regulatory proteins modulating the permeability of the membrane of mitochondrion. The Bcl-2 family of proteins are encoded by genes located on human chromosome 13 and are highly conserved in nature. The Bcl-2 family of proteins include the antiapoptotic proteins, Bcl-2, Bcl-xl, MCI-1, A1, Bcl-w and proapoptotic proteins, BAK and BAX. The Bcl-2 family of proteins received their name from the cell in which they were first discovered, a B cell leukemia.

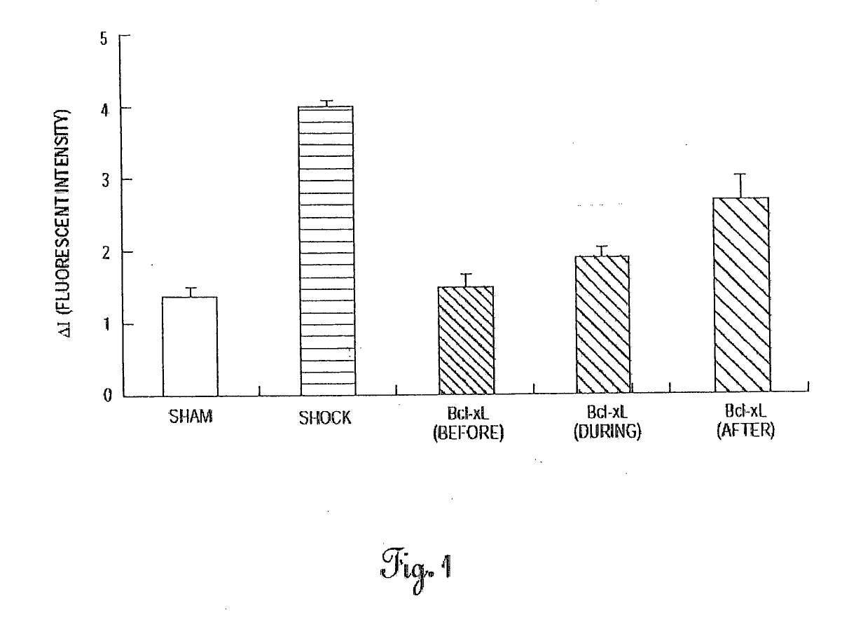

In some embodiments of the invention disclosed herein, the Bcl-2 family of proteins, and BCL-xl in particular, is used to prevent or attenuate endothelial cell dysfunction. This attenuation of apoptosis in endothelial cells maintains the fluid barrier provided by the endothelial cells and prevents or moderates the development of edema through vascular hyperpermeability. In one embodiment of the invention disclosed herein, Sprague-Dawley rats were anesthetized with urethane. Hemorrhagic shock was induced in the anesthetized rats by withdrawing blood to reduce the mean arterial pressure to 40 mm Hg for one hour. The rats were then resuscitated to 90 mmHg by administration of the shed blood and normal saline. Albumin labeled with fluorescein isothiocyanate (FITC) was given intravenously during the period in which shock was present. The mesenteric postcapillary venules in a transilluminated segment of small intestine were examined to quantitate changes in albumin flux using intravital microscopy. Recombinant Bcl-xl was suspended in a standard transfection vector and was given intravenously in an amount of approximately 2.5 microgram/ml of the total rat blood volume, before, during or after hemorrhagic shock in three separate groups of rats to determine endothelial cell integrity. Cytosolic cytochrome c levels and caspase-3 activity were also determined in mesenteric tissue collected from the animals after Bcl-xl transfection and hemorrhagic shock. As shown in FIG. 1, the administration of the protein Bcl-xl to the traumatized rats attenuated the degree of hemorrhagic shock-induced hyperpermeability. The degree of attenuation in hyperpermeability afforded by administration of Bcl-xl was greatest when Bcl-xl was administered prior to the onset of shock. Treatment of rats with Bcl-xl during the course of induced hemorrhagic shock resulted in a greater decrease in vascular hyperpermeability than did treatment with Bcl-xl after the shock period was over. A mechanism of action of the Bcl-2 family of proteins in general, and Bcl-xl, in particular, is to prevent release of cytochrome c from the mitochondrion following the onset of hemorrhagic shock. Preventing the release of cytochrome c from the mitochondria breaks the pathway to apoptosis resulting in prevention of injury to endothelial cells. Prevention of injury to endothelial cells results in an attenuation of vascular hyperpermeability during periods of hemorrhagic shock.

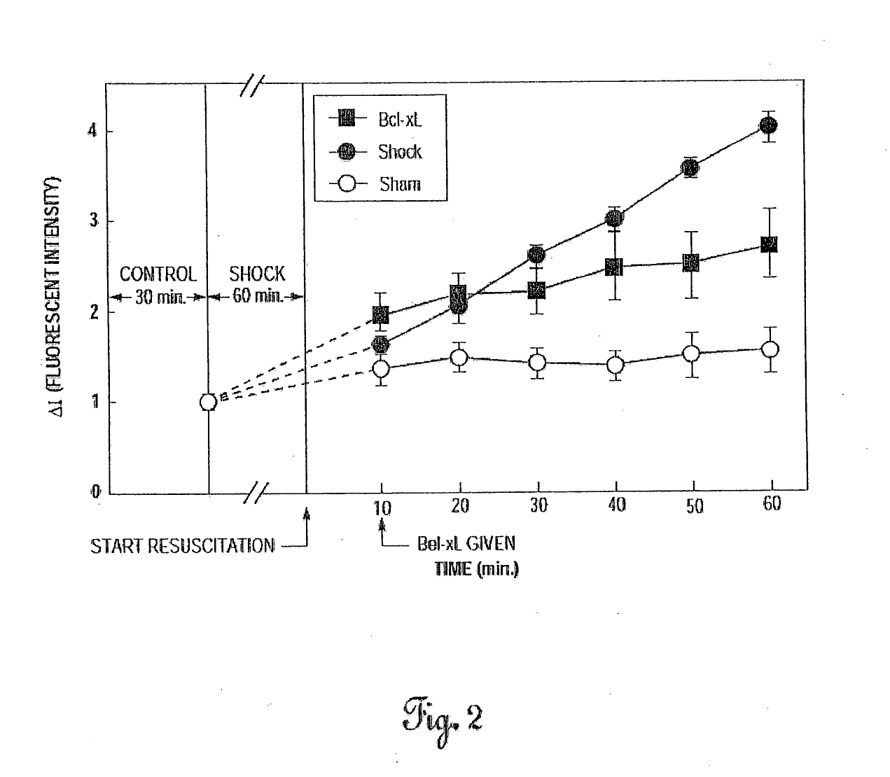

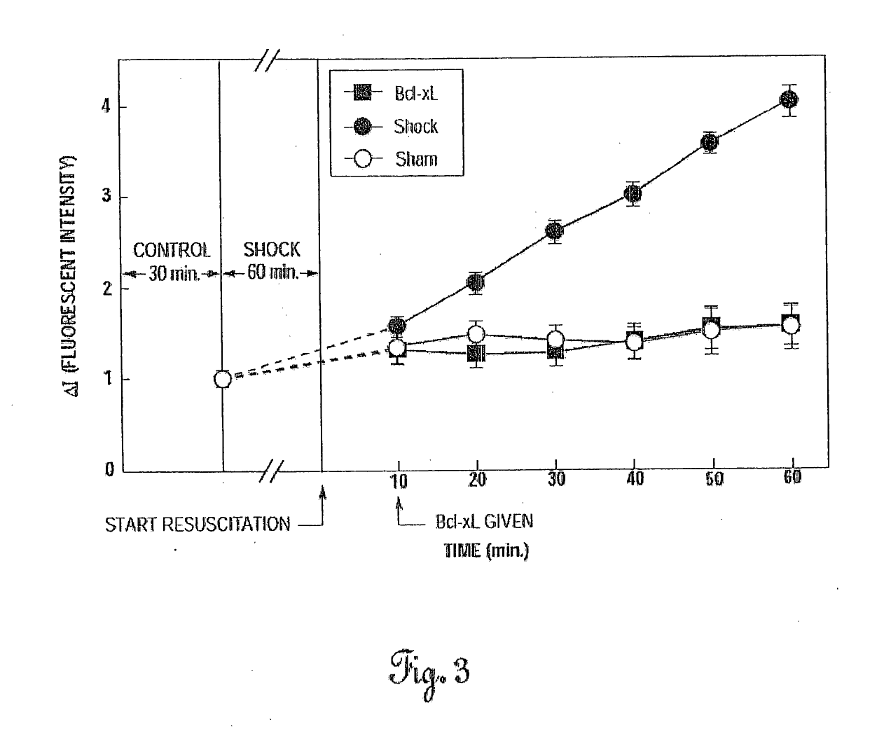

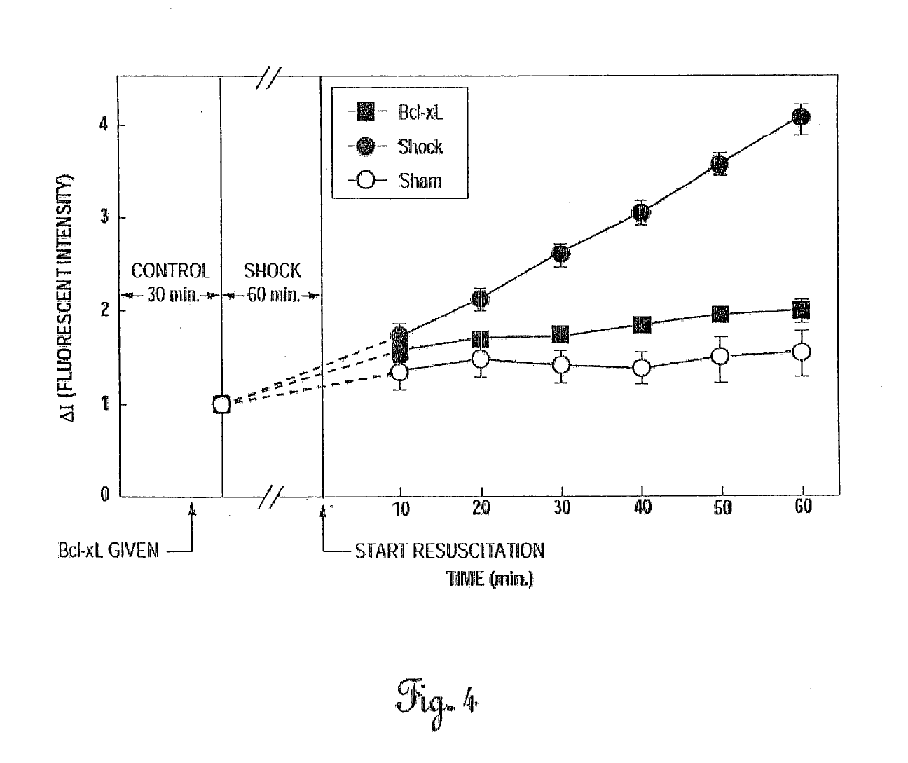

In another embodiment, Bcl-xl given after one hour of shock and 10 minutes of resuscitation attenuated vascular hyperpermeability as compared to untreated animals as shown in FIG. 2. This finding confirms that intravenous administration of the intrinsic mitochondrial regulatory protein, Bcl-xl, after the onset of shock, can diminish the amount of vascular hyperpermeability. In another embodiment of the invention disclosed herein and demonstrated in FIG. 3, administration of Bcl-xl during the period of shock, but before resuscitation efforts are started, almost eliminated the hemorrhagic shock-induced hyperpermeability. In addition, Bcl-xl was given after the shock period during resuscitation and effectively reversed the hyperpermeability induced by hemorrhagic shock. These findings support the use of the intrinsic mitochondrial regulatory protein, Bcl-xl, as a “front-line” treatment of hemorrhagic shock. In yet another embodiment, hemorrhagic shock-induced hyperpermeability was almost eliminated when rats were treated with Bcl-xl prior to the onset of shock as shown in FIG. 4.

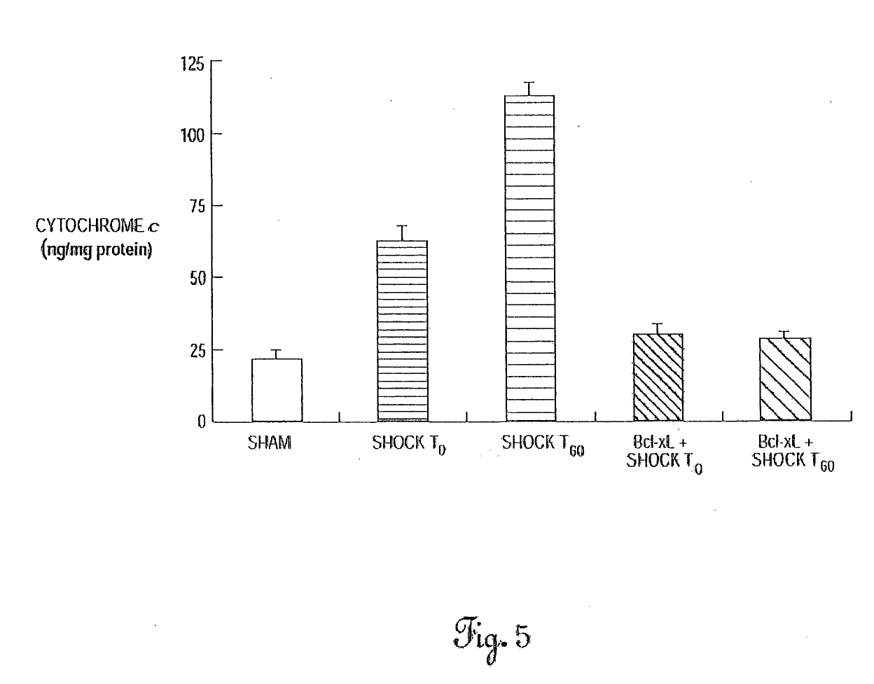

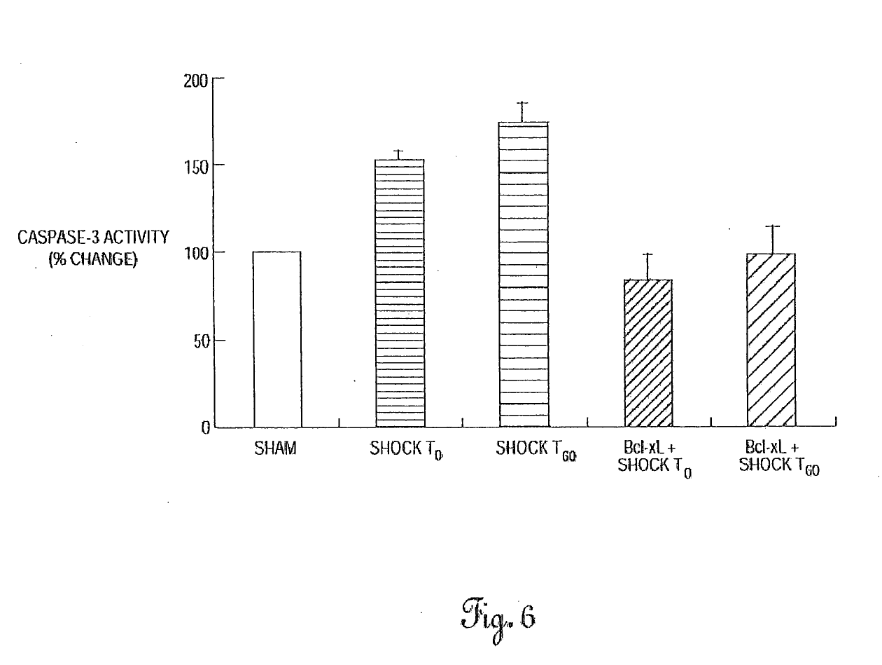

In another embodiment, the administration of Bcl-xl inhibited the release of cytochrome c into the cytoplasm from the mitochondria following hemorrhagic shock as shown in FIG. 5. FIG. 6 demonstrates another embodiment of the invention disclosed herein. Administration of Bcl-xl reduced the activation of caspase-3 following hemorrhagic shock. As described above both cytochrome c and caspase-3 play vital roles in the regulation and initiation of apoptosis of endothelial cells following hemorrhagic shock.

In the aforementioned embodiments, Bcl-xl was disclosed as having the property of inhibiting apoptosis as measured by attenuation of vascular hyperpermeability, a decrease in cytochrome c release and reduction in caspase-3 activity following administration of Bcl-xl. The use of Bcl-xl to prevent or diminish the degree of edema following trauma in mammals is clearly indicated. The other members of the Bcl-2 family of proteins, such as BAX, BAK, MCL-1, A1 and BCL-W may also have useful properties of preventing edema as does Bcl-xl and are specifically disclosed as such, herein.

The protein Bcl-xl was administered to the test animals in the aforementioned embodiments by transfection. Standard transfection vectors such as “transIT” and “chariot” are useful in facilitating entry of the intrinsic mitochondrial regulatory proteins and other substances which are disclosed herein through the membrane of the endothelial cell into the cytoplasm of the endothelial cell where regulation of apoptosis at the level of the mitochondrion can take place. Transfection vectors other than “transIT” or “chariot” are effective in promoting entry of the apoptotic modulators disclosed herein into the cytoplasm of the endothelial cell. The use of transfection to deliver Bcl-xl to the test animals was not meant to exclude other methods of delivery that are well known to those of ordinary skill in the art. For example, the intrinsic mitochondrial regulatory proteins could be bound to antibody or antigen-recognizing fragments of antibody which are specifically directed to receptor proteins on the cell membrane of endothelial cells. In this manner, the intrinsic mitochondrial regulatory protein could be delivered directly to the endothelial cell. Other delivery methods include, but are not limited to, plasmid vectors, viral vectors, liposomes, antibody vectors, and others which are included in this disclosure as if specifically set forth.

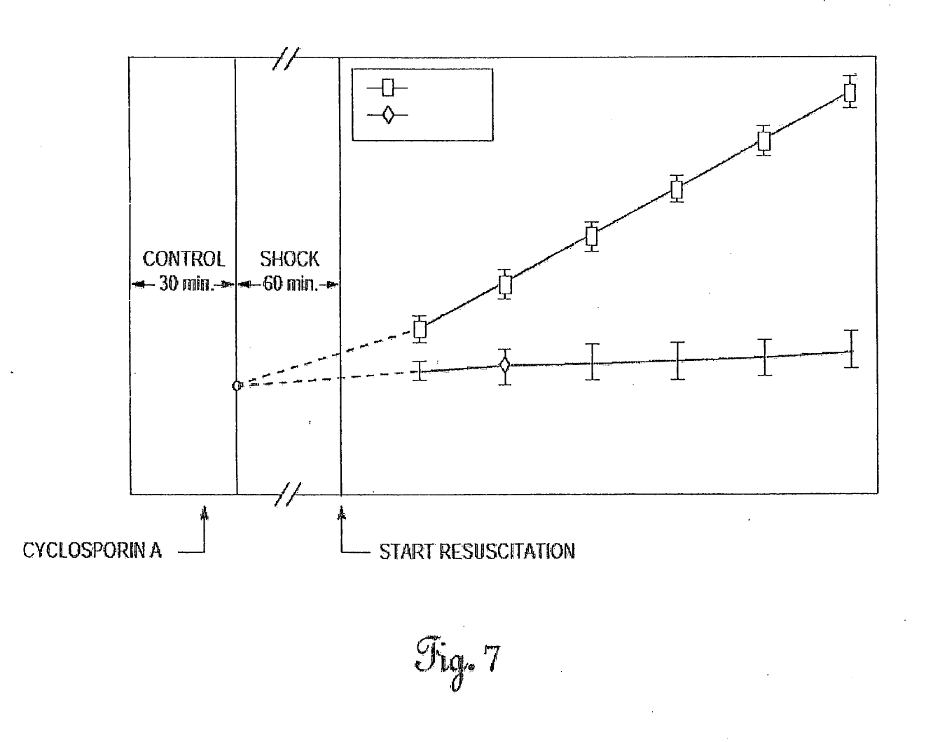

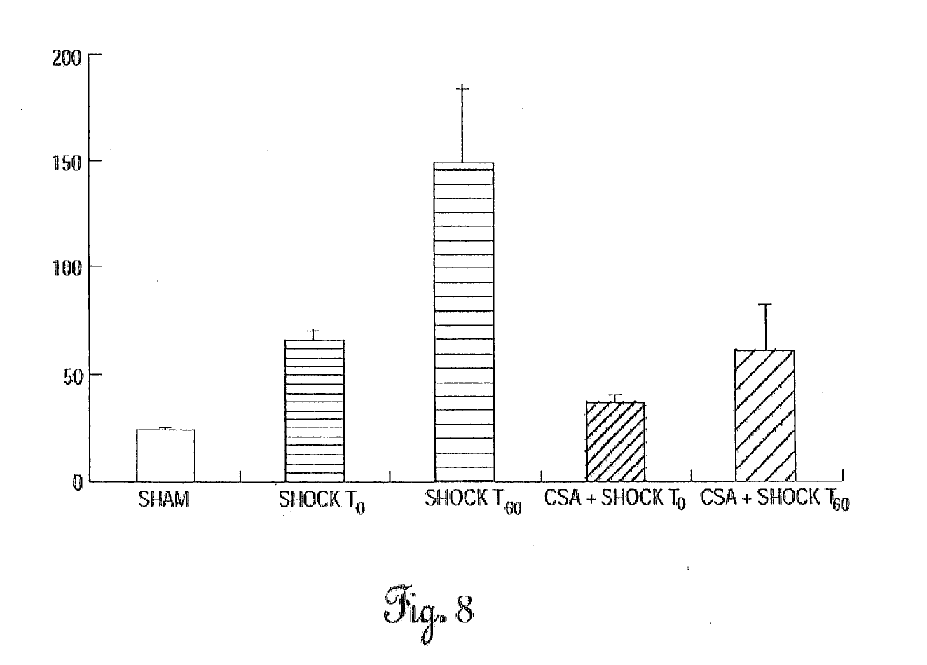

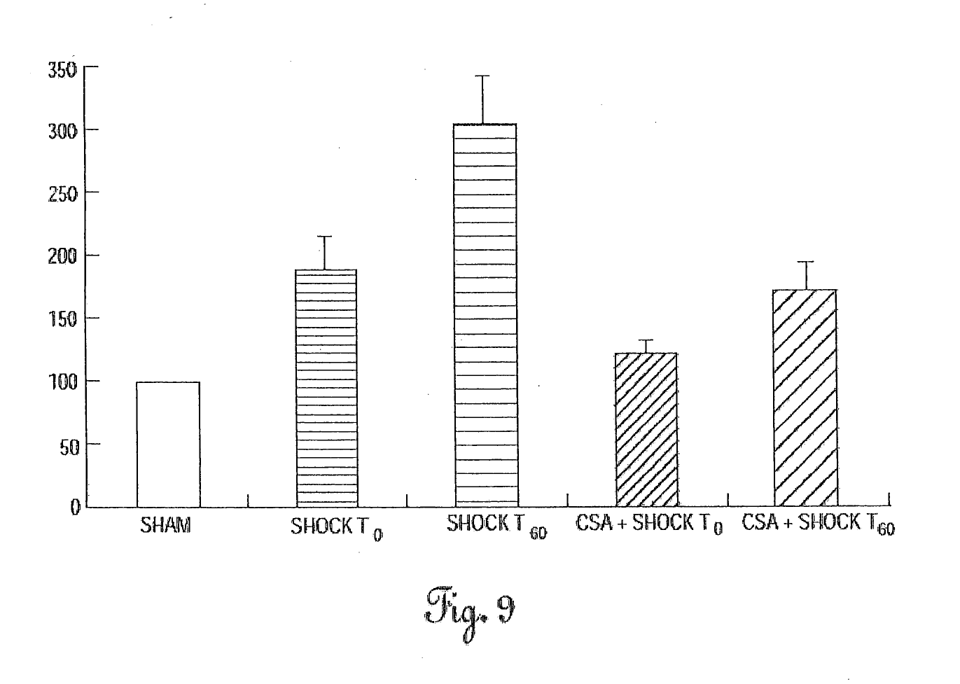

Mediators of the immune response such as cyclosporin A used initially to prevent rejection of transplanted organs, also affect apoptosis of endothelial cells as shown in FIGS. 7, 8 and 9. In this embodiment of the invention disclosed herein, the administration of cyclosporin A by transfection, for example, prior to the induction of shock in rats as described above, resulted in a complete elimination of vascular hyperpermeability as shown in FIG. 7. That cyclosporin A exerts its effect on vascular hyperpermeability by inhibiting apoptosis of endothelial cells is shown in FIG. 8 and FIG. 9 wherein administration of cyclosporin A inhibits cytochrome c release from mitochondria and diminishes the induction of caspase-3 activity by hemorrhagic shock, respectively. Cyclosporin A is effective in preventing edema in mammals following acute trauma. The amount of cyclosporine A administered to traumatized animals is an amount which effectively inhibits apoptosis and is in a range of approximately 5 microliters to approximately 20 microliters per milliliter of blood volume.

Because of the role of ROS in the development of cell permeability following hemorrhagic shock, antioxidants were employed to inhibit the development of ROS and minimize the development of cell permeability and cell injury related to the development of ROS during apoptosis. In this embodiment of the invention disclosed herein, antioxidants such as alpha-lipoic acid were administered to animals traumatized as described above. The administration of alpha-lipoic acid attenuated the amount of vascular hyperpermeability induced by hemorrhagic shock-induced apoptosis. Alpha-lipoic acid administered by transfection in a dosage of about 100 mg/kg was effective in reducing the amount of vascular hyperpermeability if administered either before the onset of hemorrhagic shock or within 60 minutes after the development of hemorrhagic shock.

In another embodiment of the invention described herein, it is disclosed that angiopoietin-1, an endothelial cell growth factor, administered to mammals with hemorrhagic shock, attenuated the amount of vascular hyperpermeability demonstrated by those traumatized animals. Angiopoietin-1 administered intravenously in a dosage of 200 ng/ml to traumatized animals attenuated the amount of vascular hyperpermeability observed in those animals. The effect of angiopoietin-1 on lessening vascular hyperpermeability was to disrupt the apoptotic signaling mechanism which initiates and sustains the process of apoptosis by inhibiting one or a combination of factors comprising: (1) BAK peptide-induced collapse of mitochondrial transmembrane potential, (2) second mitochondrial derived activator of caspases release (smac), (3) cytochrome c release, and (4) activation of caspase-3.

As described above, intrinsic mitochondrial regulatory proteins were administered intravenously to traumatized animals. It is further disclosed herein that the intrinsic mitochondrial regulatory proteins may be administered by the sublingual route, direct injection into a body cavity or through the peritoneum into the abdominal cavity. Administration of the intrinsic mitochondrial regulatory proteins by these other avenues would raise the threshold of apoptosis and prevent vascular hyperpermeability and edema.

When foreign proteins are injected into a mammal, the host animal recognizes the proteins as foreign and attempts to eliminate them quickly from the body of the host. This rapid elimination of these administered proteins can diminish the activity of those administered proteins and deprive the host animal with their full benefit. This removal of administered proteins can be inhibited to some extent by binding to the foreign proteins substances which slow or prevent the process of natural elimination of foreign proteins. It is specifically disclosed herein, that the intrinsic mitochondrial regulatory proteins can be specifically attached to other compounds prior to administration to the traumatized animal which prolongs the effective time period in which the intrinsic mitochondrial regulatory protein can act to inhibit apoptosis in endothelial cells of traumatized animals. Those substances which can be attached to the intrinsic mitochondrial regulatory proteins to prolong their presence in the animal's circulation include but are not limited to sugars, carbohydrates, nucleotides, polyethylene glycol and the like.

The invention disclosed herein is a method for treatment of patients with edema following the development of shock. The method comprises modulating the apoptotic process in the endothelial cells lining the lumen of small venules, capillaries and other vascular structures, in order to preserve the barrier to leakage of fluid from the blood to the other tissues and prevent or diminish edema. This amelioration of edema would prevent organ failure and promote the effectiveness of resuscitation measures used to treat shock. As shown above, regulatory proteins, pharmaceuticals, antioxidants, endothelial growth factors, and other compounds and processes related to regulation of apoptosis can be modulated to prevent the death of endothelial cells and development of edema. In particular and in various embodiments, manipulation of the Bcl-2 family of proteins, immunomodulating compounds such as cyclosporin A, endothelial growth factors such as angiopoietin-1, and antioxidants provide such desirable results. Administration of such compounds, either alone or in combination, to trauma patients would save many lives and prevent other morbidities caused by the organ damage associated with edema resulting from vascular hyperpermeability. Administration of a combination of the apoptotic modulators described above would inhibit the apoptotic cascade at different points making the use of a combination of the aforementioned apoptotic modulators an effective inhibitor of vascular permeability caused by endothelial cell death.

Given the millions of compounds, combinations, and processes that affect apoptosis and the teachings in the current application, it should be understood that one of skill in the art may identify alternative embodiments to those specifically named herein. Thus, the embodiments described above are not intended to limit the breadth of the claims to follow; the broadest aspect of the current application being modulation of apoptosis to treat hyperpermeability and related conditions that normally result from hemorrhagic shock.

We claim:

1. A method for attenuating conditions associated with hyperpermeability caused by hemorrhagic shock in mammals comprising raising the threshold for onset of apoptotic processes resultant from the hemorrhagic shock whereby the conditions are attenuated.

2. The method of claim 1 wherein raising the threshold for onset of apoptotic processes comprises:
preparing in deliverable form a composition comprising anantioxidant, pharmaceutical, regulatory protein, intrinsic mitochondrial regulatory protein, endothelial growth factor, or combinations thereof; and
administering an effective amount of said composition to a mammal so as to raise the threshold of apoptosis.

3. The method of claim 2 wherein the composition comprises alpha-lipoic acid, cyclosporine-A, angiopoietin-1, a member of the Bcl-2 family of proteins, non-Bcl-2 proteins, or combinations thereof.

4. The method of claim 1 wherein conditions comprise trauma, organ trauma, infections, inflammation, degenerative disease, edema and other conditions associated with hemorrhagic shock.

5. The method of claim 2 wherein preparing in deliverable form comprises mixing with a transfection vector, binding to another compound, attaching to an antibody, or combinations thereof.

6. The method for attenuation of vascular hyperpermeability caused by hemorrhagic shock in mammals as defined in claim 2 wherein said effective amount of said antioxidant is about 100 micromole/liter.

7. The method for attenuation of vascular hyperpermeability caused by hemorrhagic shock in mammals as defined in claim 1 wherein said mammals are human beings.

8. The method for attenuation of vascular hyperpermeability caused by hemorrhagic shock in mammals as defined in claim 2 wherein said effective amount of a pharmaceutical is an effective amount to inhibit apoptosis.

9. The method for attenuation of vascular hyperpermeability caused by hemorrhagic shock in mammals as defined in claim 2 wherein said effective amount of said endothelial growth factor is about 200 nanograms/milliliter.

10. The method for attenuation of vascular hyperpermeability caused by hemorrhagic shock in mammals as defined in claim 2 wherein said intrinsic mitochondrial regulatory protein from the Bcl-2 family of proteins comprises: Bcl-2, Bcl-xl, MCI-1, A1, Bcl-w, or combinations thereof.

11. The method for attenuation of vascular hyperpermeability caused by hemorrhagic shock in mammals as defined in claim 2 wherein said effective amount of said intrinsic mitochondrial protein to raise the threshold for apoptosis caused by hemorrhagic shock is at least 2.5 micrograms/milliliter.

12. A method for prevention of endothelial cell injury associated with hemorrhagic shock in mammals comprising steps of:
preparation of an antioxidant in deliverable form;
administering an effective amount of said antioxidant to said mammals so as to raise the threshold for apoptosis in endothelial cells;
whereby said endothelial cell injury associated with hemorrhagic shock is prevented.

13. The method for prevention of endothelial cell injury associated with hemorrhagic shock in mammals as defined in claim 12 wherein said antioxidant is alpha-lipoic acid.

14. The method for prevention of endothelial cell injury associated with hemorrhagic shock in mammals as defined in claim 13 wherein said effective amount of alpha-lipoic acid is about 100 micromole/liter.

15. The method for prevention of endothelial cell injury associated with hemorrhagic shock in mammals as defined in claim 12 wherein said deliverable form is said antioxidant in a mixture with a transfection vector.

16. The method for prevention of endothelial cell injury associated with hemorrhagic shock in mammals as defined in claim 12 wherein said mammals are human beings.

17. A method for prevention of endothelial cell injury associated with hemorrhagic shock in mammals comprising steps of:
preparing a pharmaceutical product in a deliverable form;
administering said pharmaceutical product in an effective amount to raise the threshold of apoptosis in endothelial cells;
whereby endothelial cell injury is prevented.

18. The method for prevention of endothelial cell injury associated with hemorrhagic shock in mammals as defined in claim 17 wherein said pharmaceutical product is cyclosporine-A.

19. The method for prevention of endothelial cell injury associated with hemorrhagic shock in mammals as defined in claim 18 wherein said effective amount of cyclosporine-A is in a range of approximately 5 microliters to 20 microliters per milliliter of blood volume.

20. The method for prevention of endothelial cell injury associated with hemorrhagic shock in mammals as defined in claim 17 wherein said deliverable form is said pharmaceutical product in a mixture with a transfection vector.

21. The method for prevention of endothelial cell injury associated with hemorrhagic shock in mammals as defined in claim 17 wherein said mammal is a human being.

22. A method for prevention of endothelial cell injury associated with hemorrhagic shock in mammals comprising steps of:
preparing an endothelial growth factor in a deliverable form;
administering said endothelial growth factor in an effective amount to increase the threshold for apoptosis in endothelial cells;
whereby endothelial cell injury associated with hemorrhagic shock is prevented.

23. The method for prevention of endothelial cell injury associated with hemorrhagic shock in mammals as defined in claim 22 wherein said endothelial growth factor is angiopoietin-1.

24. The method for prevention of endothelial cell injury associated with hemorrhagic shock in mammals as defined in claim 23 wherein said effective amount of angiopoietin-1 is about 200 nanograms/milliliter.

25. The method for prevention of endothelial cell injury associated with hemorrhagic shock in mammals as defined in claim 22 wherein said deliverable form is said endothelial growth factor in a mixture with a transfection vector.

26. The method for prevention of endothelial cell injury associated with hemorrhagic shock in mammals as defined in claim 22 wherein said mammal is a human being.

27. A method for prevention of endothelial cell injury associated with hemorrhagic shock in mammals comprising steps of:
preparing an intrinsic mitochondrial regulatory protein in a deliverable form;
administering said intrinsic mitochondrial regulatory protein in an effective amount to raise the threshold for apoptosis in endothelial cells;
whereby the endothelial cell injury caused by hemorrhagic shock is prevented.

28. The method for prevention of endothelial cell injury associated with hemorrhagic shock in mammals as defined in claim 27 wherein said intrinsic mitochondrial regulatory protein is Bcl-2.

29. The method for prevention of endothelial cell injury associated with hemorrhagic shock in mammals as defined in claim 27 wherein said intrinsic mitochondrial regulatory protein is Bcl-xl.

30. The method for prevention of endothelial cell injury associated with hemorrhagic shock in mammals as defined in claim 27 wherein said intrinsic mitochondrial regulatory protein is MCI-1.

31. The method for prevention of endothelial cell injury associated with hemorrhagic shock in mammals as defined in claim 27 wherein said intrinsic mitochondrial regulatory protein is A1.

32. The method for prevention of endothelial cell injury associated with hemorrhagic shock in mammals as defined in claim 27 wherein said intrinsic mitochondrial regulatory protein is Bcl-w.

33. The method for prevention of endothelial cell injury associated with hemorrhagic shock in mammals as defined in claim 27 wherein said deliverable form is said intrinsic mitochondrial regulatory protein in a mixture with a transfection vector.

34. The method for prevention of endothelial cell injury associated with hemorrhagic shock defined in claim 27 wherein said effective amount of intrinsic mitochondrial regulatory protein to raise the threshold for apoptosis caused by hemorrhagic shock is at least 2.5 micrograms/milliliter.

35. The method for prevention of endothelial cell injury associated with hemorrhagic shock in mammals as defined in claim 27 wherein said mammal is a human being.

36. A method for inhibiting the release of cytochrome c from the mitochondria of endothelial cells during hemorrhagic shock comprising steps of:
preparing an intrinsic mitochondrial regulatory protein in a deliverable form;
administering said intrinsic mitochondrial regulatory protein in an effective amount to raise the threshold for apoptosis in a mammal with hemorrhagic shock;
whereby the release of cytochrome c from endothelial cells during hemorrhagic shock is diminished.

37. The method for inhibiting the release of cytochrome c from the mitochondria of endothelial cells during hemorrhagic shock in mammals as defined in claim 36 wherein said intrinsic mitochondrial regulatory protein is Bcl-2.

38. The method for inhibiting the release of cytochrome c from the mitochondria of endothelial cells during hemorrhagic shock in mammals as defined in claim 36 wherein said intrinsic mitochondrial regulatory protein is Bcl-xl.

39. The method for inhibiting the release of cytochrome c from the mitochondria of endothelial cells during hemorrhagic shock in mammals as defined in claim 36 wherein said intrinsic mitochondrial regulatory protein is MCI-1.

40. The method for inhibiting the release of cytochrome c from the mitochondria of endothelial cells during hemorrhagic shock in mammals as defined in claim 36 wherein said intrinsic mitochondrial regulatory protein is A1.

41. The method for inhibiting the release of cytochrome c from the mitochondria of endothelial cells during hemorrhagic shock in mammals as defined in claim 36 wherein said intrinsic mitochondrial regulatory protein is Bcl-w.

42. The method for inhibiting the release of cytochrome c from the mitochondria of endothelial cells during hemorrhagic shock in mammals as defined in claim 36 wherein said deliverable form is said intrinsic mitochondrial regulatory protein in a mixture with a transfection vector.

43. The method for inhibiting the release of cytochrome c from the mitochondria of endothelial cells during hemorrhagic shock in mammals as defined in claim 36 wherein said effective amount of intrinsic mitochondrial regulatory protein to raise the threshold for apoptosis caused by hemorrhagic shock is at least 2.5 micrograms/milliliter.

44. The method for inhibiting the release of cytochrome c from the mitochondria of endothelial cells during hemorrhagic shock in mammals as defined in claim 36 wherein said mammals are human beings.

45. A method for inhibiting induction of caspase-3 from the mitochondria of endothelial cells following hemorrhagic shock in mammals comprising steps of:
preparing a pharmaceutical product which inhibits induction of caspase-3 in a deliverable form;
administering said pharmaceutical product in an effective amount to inhibit the induction of caspase-3 from the mitochondria of endothelial cells following hemorrhagic shock;
whereby preventing apoptosis in endothelial cells caused by hemorrhagic shock.

46. The method for inhibiting induction of caspase-3 from the mitochondria of endothelial cells following hemorrhagic shock in mammals as defined in claim 45 wherein said pharmaceutical product is cyclosporine-A.

47. The method for inhibiting induction of caspase-3 from the mitochondria of endothelial cells following hemorrhagic shock in mammals as defined in claim 46 wherein said effective amount of cyclosporine-A is in a range of approximately 5 microliters to 20 microliters per milliliter.

48. The method for inhibiting induction of caspase-3 from the mitochondria of endothelial cells following hemorrhagic shock in mammals as defined in claim 45 wherein said deliverable form is said pharmaceutical agent which inhibits induction of caspase-3 in a mixture with a transfection vector.

49. The method for inhibiting induction of caspase-3 from the mitochondria of endothelial cells following hemorrhagic shock in mammals as defined in claim 45 wherein said mammals are human beings.

50. A method for inhibiting apoptotic signaling in mitochondria of endothelial cells associated with hemorrhagic shock in mammals comprising steps of:
preparing an endothelial growth factor in a deliverable form;
administering said endothelial growth factor in an effective amount to inhibit apoptotic signaling in mitochondria of endothelial cells;
whereby endothelial cell injury associated with hemorrhagic shock is prevented.

51. The method for inhibiting apoptotic signaling in mitochondria of endothelial cells associated with hemorrhagic shock in mammals as defined in claim 50 wherein said endothelial growth factor is angiopoietin-1.

52. The method for inhibiting apoptotic signaling in mitochondria of endothelial cells associated with hemorrhagic shock in mammals as defined in claim 51 wherein said effective amount of said angiopoietin-1 is about 200 nanograms/milliliter.

53. The method for inhibiting apoptotic signaling in mitochondria of endothelial cells associated with hemorrhagic shock in mammals as defined in claim 50 wherein said deliverable form is said endothelial growth factor in a mixture with a transfection vector.

54. The method for inhibiting apoptotic signaling in mitochondria of endothelial cells associated with hemorrhagic shock in mammals as defined in claim 50 wherein said mammals are human beings.

55. A method for inhibiting the development of reactive oxygen species by mitochondria of endothelial cells following hemorrhagic shock in mammals comprising steps of:
preparation of an antioxidant in deliverable form;
administering an effective amount of said antioxidant to said mammal so as to prevent the development of reactive oxygen species by the mitochondria of endothelial cells;
whereby endothelial cell injury is prevented.

56. The method for inhibiting the development of reactive oxygen species by mitochondria of endothelial cells following hemorrhagic shock in mammals as defined in claim 55 wherein said antioxidant is alpha-lipoic acid.

57. The method for inhibiting the development of reactive oxygen species by mitochondria of endothelial cells following hemorrhagic shock in mammals as defined in claim 56 wherein said effective amount of said alpha lipoic acid is about 100 micromole/liter.

58. The method for inhibiting the development of reactive oxygen species by mitochondria of endothelial cells following hemorrhagic shock in mammals as defined in claim 55 wherein said deliverable form is said antioxidant in a mixture with a transfection vector.

59. The method for inhibiting the development of reactive oxygen species by mitochondria of endothelial cells following hemorrhagic shock in mammals as defined in claim 55 wherein said mammals are human beings.

60. A method for attenuation of BAK peptide-induced collapse of mitochondrial transmembrane potential caused by hemorrhagic shock in mammals comprising steps of:
preparing an endothelial growth factor in a deliverable form;
administering said endothelial growth factor in an effective amount to attenuate the BAK peptide-induced collapse of mitochondrial transmembrane potential in mammals with hemorrhagic shock;
whereby vascular hyperpermeability caused by hemorrhagic shock is diminished.

61. The method for attenuation of BAK peptide-induced collapse of mitochondrial transmembrane potential caused by hemorrhagic shock in mammals as defined in claim 60 wherein said endothelial growth factor is angiopoietin-1.

62. The method for attenuation of BAK peptide induced collapse of mitochondrial transmembrane potential caused by hemorrhagic shock in mammals as defined in claim 61 wherein said effective amount of said angiopoietin-1 is about 200 nanograms/milliliter.

63. The method for attenuation of BAK peptide-induced collapse of mitochondrial transmembrane potential caused by hemorrhagic shock in mammals as defined in claim 60 wherein said deliverable form is said endothelial growth factor in a mixture with a transfection vector.

64. The method for attenuation of BAK peptide-induced collapse of mitochondrial transmembrane potential caused by hemorrhagic shock in mammals as defined in claim 60 wherein said mammals are human beings.

65. A method for attenuation of the second mitochondrial derived activator of caspases release (smac) caused by hemorrhagic shock in mammals comprising steps of:
preparing an endothelial growth factor in a deliverable form;
administering said endothelial growth factor in an effective amount to attenuate the second mitochondrial derived activator of caspases release (smac) in mammals with hemorrhagic shock;
whereby said second mitochondrial derived activator of caspases release (smac) caused by hemorrhagic shock is diminished.

66. The method for attenuation of second mitochondrial derived activator of caspases release (smac) caused by hemorrhagic shock in mammals as defined in claim 65 wherein said endothelial growth factor is angiopoietin-1.

67. The method for attenuation of second mitochondrial derived activator of caspases release (smac) in mammals with hemorrhagic shock as defined in claim 66 wherein said effective amount of said angiopoietin-1 is about 200 nanograms/milliliter.

68. The method for attenuation of second mitochondrial derived activator of caspases release (smac) caused by hemorrhagic shock in mammals as defined in claim 65 wherein said deliverable form is said endothelial growth factor in a mixture with a transfection vector.

69. The method for attenuation of second mitochondrial derived activator of caspases release (smac) caused by hemorrhagic shock in mammals as defined in claim 65 wherein said mammals are human beings.

70. A method for inhibition of cytochrome c release caused by hemorrhagic shock in mammals comprising steps of:
preparing an endothelial growth factor in a deliverable form;
administering said endothelial growth factor in an effective amount to inhibit the release of cytochrome c in mammals with hemorrhagic shock;
whereby vascular hyperpermeability caused by hemorrhagic shock is diminished.

71. The method for inhibition of cytochrome c release caused by hemorrhagic shock in mammals as defined in claim 70 wherein said endothelial growth factor is angiopoietin-1.

72. The method for inhibition of cytochrome c release caused by hemorrhagic shock in mammals as defined in claim 71 wherein said effective amount of said angiopoietin-1 is about 200 nanograms/milliliter.

73. The method for inhibition of cytochrome c release caused by hemorrhagic shock in mammals as defined in claim 70 wherein said deliverable form is said endothelial growth factor in a mixture with a transfection vector.

74. The method for inhibition of cytochrome c release caused by hemorrhagic shock in mammals as defined in claim 70 wherein said mammals are human beings.

75. A method for inhibition of caspase-3 activation caused by hemorrhagic shock in mammals comprising steps of:
preparing an endothelial growth factor in a deliverable form;
administering said endothelial growth factor in an effective amount to inhibit the activation of caspase-3 in mammals with hemorrhagic shock;
whereby vascular hyperpermeability caused by hemorrhagic shock is diminished.

76. The method for inhibition of caspase-3 activation caused by hemorrhagic shock in mammals as defined in claim 75 wherein said endothelial growth factor is angiopoietin-1.

77. The method for inhibition of caspase-3 activation caused by hemorrhagic shock in mammals as defined in claim 76 wherein said effective amount of said angiopoietin-1 is about 200 nanograms/milliliter.

78. The method for inhibition of caspase-3 activation caused by hemorrhagic shock in mammals as defined in claim 75 wherein said deliverable form is said endothelial growth factor in a mixture with a transfection vector.

79. The method for inhibition of caspase-3 activation caused by hemorrhagic shock in mammals as defined in claim 75 wherein said mammals are human beings.

80. A method for attenuation of vascular hyperpermeability caused by hemorrhagic shock comprising steps of:
Preparing any combination of one or more endothelial growth factors, pharmaceutical agent, antioxidant, and/or intrinsic mitochondrial regulatory protein in a deliverable form;
Administering said combination of endothelial growth factors, pharmaceutical agent, antioxidant and/or intrinsic mitochondrial regulatory protein in an effective amount;
Whereby said vascular hyperpermeability is diminished.

81. The method of claim 80 for attenuation of vascular hyperpermeability caused by hemorrhagic shock wherein said endothelial growth factor is angiopoietin-1.

82. The method for attenuation of vascular hyperpermeability caused by hemorrhagic shock as defined in claim 80 wherein said pharmaceutical agent is cyclosporine-A.

83. The method for attenuation of vascular hyperpermeability caused by hemorrhagic shock as defined in claim 80 wherein said antioxidant is alpha-lipoic acid.

84. The method for attenuation of vascular hyperpermeability caused by hemorrhagic shock as defined in claim 80 wherein said intrinsic mitochondrial regulatory protein is Bcl-2.

85. The method for attenuation of vascular hyperpermeability caused by hemorrhagic shock as defined in claim 80 wherein said intrinsic mitochondrial regulatory protein is Bcl-xl.

86. The method for attenuation of vascular hyperpermeability caused by hemorrhagic shock as defined in claim 80 wherein said intrinsic mitochondrial regulatory protein is MCI-1.

87. The method for attenuation of vascular hyperpermeability caused by hemorrhagic shock as defined in claim 80 wherein said intrinsic mitochondrial regulatory protein is A1.

88. The method for attenuation of vascular hyperpermeability caused by hemorrhagic shock as defined in claim 80 wherein said intrinsic mitochondrial regulatory protein is Bcl-w.

89. A method for prevention of endothelial cell injury caused by hemorrhagic shock comprising steps of:
preparing any combination of one or more endothelial growth factors, pharmaceutical agent, antioxidant, and/or intrinsic mitochondrial regulatory protein in a deliverable form;
administering said combination of endothelial growth factor, pharmaceutical agent, antioxidant and/or intrinsic mitochondrial regulatory protein in an effective amount;
whereby endothelial cell injury due to hemorrhagic shock is prevented.

90. The method for prevention of endothelial cell injury caused by hemorrhagic shock as defined in claim 89 wherein said endothelial growth factor is angiopoietin-1.

91. The method for prevention of endothelial cell injury caused by hemorrhagic shock as defined in claim 89 wherein said pharmaceutical agent is cyclosporine-A.

92. The method for prevention of endothelial cell injury caused by hemorrhagic shock as defined in claim 89 wherein said antioxidant is alpha-lipoic acid.

93. The method for prevention of endothelial cell injury caused by hemorrhagic shock as defined in claim 89 wherein said intrinsic mitochondrial regulatory proteins is Bcl-2.

94. The method for prevention of endothelial cell injury caused by hemorrhagic shock as defined in claim 89 wherein said intrinsic mitochondrial regulatory protein is Bcl-xl.

95. The method for prevention of endothelial cell injury caused by hemorrhagic shock as defined in claim 89 wherein said intrinsic mitochondrial regulatory protein is MCI-1.

96. The method for prevention of endothelial cell injury caused by hemorrhagic shock as defined in claim 89 wherein said intrinsic mitochondrial regulatory protein is A1.

97. The method for prevention of endothelial cell injury caused by hemorrhagic shock as defined in claim 89 wherein said intrinsic mitochondrial regulatory protein is Bcl-w.

98. A method for prolonging an effective therapeutic time period of intrinsic mitochondrial regulatory proteins to raise the threshold of apoptosis in mammals with hemorrhagic shock comprising steps of:
binding said intrinsic mitochondrial regulatory proteins to another compound;
administering said intrinsic mitochondrial regulatory protein bound to another compound to said mammal in hemorrhagic shock;
whereby said intrinsic mitochondrial regulatory proteins bound to another compound attenuates apoptosis of endothelial cells for a longer period of time resulting in diminished vascular hyperpermeability.

99. The method for prolonging the effective therapeutic time period of intrinsic mitochondrial regulatory proteins to raise the threshold of apoptosis in mammals with hemorrhagic shock as defined in claim 98 wherein said intrinsic mitochondrial regulatory protein comprises Bcl-2, Bcl-xl, MCI-1, A1, Bcl-w, or combinations thereof.

100. The method for prolonging the effective therapeutic time period for intrinsic mitochondrial regulatory proteins to raise the threshold of apoptosis in mammals with hemorrhagic shock as defined in claim 98 wherein said compound to be bound to said intrinsic mitochondrial regulatory proteins comprises sugars, carbohydrates, nucleotides and polyethylene glycol, or combinations thereof.

101. The method for prolonging effective therapeutic time period for intrinsic mitochondrial regulatory proteins to raise the threshold of apoptosis in mammals with hemorrhagic shock as defined in claim 98 wherein said mammals are human beings.

102. A method for delivery of intrinsic mitochondrial regulatory proteins to specific receptors on cell membranes of endothelial cells of mammals with hemorrhagic shock comprising steps of:
preparing an antibody specific to said specific receptor on the cell membrane of endothelial cells;
attaching said intrinsic mitochondrial regulatory protein to said antibody;
administering said intrinsic mitochondrial regulatory protein attached to said antibody to said mammal with hemorrhagic shock:
whereby said antibody delivers said intrinsic mitochondrial regulatory protein to said specific receptor causing attenuation of apoptosis and diminished vascular hyperpermeability.

103. The method for delivery of the intrinsic mitochondrial regulatory proteins to the specific receptors on the cell membranes of endothelial cells of mammals with hemorrhagic shock as defined in claim 102 wherein said intrinsic mitochondrial regulatory protein comprises Bcl-2, Bcl-xl, MCI-1, A1, and Bcl-w, or combinations thereof.

104. The method for delivery of the intrinsic mitochondrial regulatory proteins to the specific receptors on the cell membranes of endothelial cells of mammals with hemorrhagic shock as defined in claim 102 wherein said mammals are human beings.
